(19) United States
(12) Patent Application Publication
(10) Pub. No.: US 2005/0288215 A1
(43) Pub. Date: Dec. 29, 2005

(54) Inhibition of nuclear export as a treatment for cardiac hypertrophy and heart failure

(76) Inventor: Timothy A. McKinsey, Broomfield, CO (US)

Correspondence Address: FULBRIGHT & JAWORSKI L.L.P., 600 CONGRESS AVE., SUITE 2400, AUSTIN, TX, 78701, US

(21) Appl. No.: 11/099,417

(22) Filed: Apr. 5, 2005

Related U.S. Application Data
(60) Provisional application No. 60/559,493, filed on Apr. 5, 2004.

(51) Int. Cl. A61K 38/17; A61K 31/553

(52) U.S. Cl. 514/2; 514/211.08; 514/28

(57) ABSTRACT

The present invention provides methods of treating cardiac hypertrophy by administering a drug that is known to be a non-selective inhibitor of nuclear protein export to patient in need thereof.

### BACKGROUND OF THE INVENTION

This application claims benefit of priority to U.S. Provisional Application Ser. No. 60/559,493 filed Apr. 5, 2004, the entire contents of which are hereby incorporated by reference.

1. Field of the Invention

The present invention relates generally to the fields of developmental biology and molecular biology. More particularly, it concerns gene regulation and cellular physiology in cardiomyocytes. Specifically, the invention relates to the use of inhibitors of nuclear export to treat cardiac hypertrophy and heart failure.

2. Description of Related Art

Cardiac hypertrophy in response to an increased workload imposed on the heart is a fundamental adaptive mechanism which, while beneficial in the initial stages to help compensate for the physiological problems present in the body, eventually leads to heart failure (which can also occur without hypertrophy). Hypertrophy is a specialized process reflecting a quantitative increase in cell size and mass (rather than cell number) as the result of any or a combination of neural, endocrine or mechanical stimuli. Hypertension, another factor involved in cardiac hypertrophy, is a frequent precursor of congestive heart failure. When heart failure occurs, the left ventricle usually is hypertrophied and dilated and indices of systolic function, such as ejection fraction, are reduced. Clearly, the cardiac hypertrophic response is a complex syndrome and the elucidation of the pathways leading to both cardiac hypertrophy and heart failure will be beneficial in the treatment of cardiovascular disease resulting from various stimuli.

A family of transcription factors, the myocyte enhancer factor-2 family (MEF2), is involved in cardiac hypertrophy. For example, a variety of stimuli can elevate intracellular calcium, resulting in a cascade of intracellular signaling systems or pathways, including calcineurin, CAM kinases, PKC and MAP kinases. All of these signals activate MEF2 and result in cardiac hypertrophy, and it is further known that certain histone deacetylase proteins (HDACs) are involved in modulating MEF2 activity. In order to accomplish this modulation, HDACs that bind MEF2, known as Class II HDACs, must be present in the nucleus of the cell to repress MEF2 driven transcription, and when HDACs are exported out of the nucleus in response to a variety of stimuli, MEF2 genes are activated, leading to hypertrophy and heart failure.

Eleven different HDACs have been cloned from vertebrate organisms. All share homology in the catalytic region. Histone acetylases (HATs) and HDACs play a major role in the control of gene expression. The balance between activities of HATs and HDACs determines the level of histone acetylation. Consequently, acetylated histones cause relaxation of chromatin and activation of gene transcription, whereas deacetylated chromatin is generally transcriptionally inactive. In a previous report, the inventor and others have demonstrated that HDAC 4 and 5 dimerize with MEF2 and repress the transcriptional activity of MEF2 and, further, that this interaction requires the presence of the N-terminus of the HDAC 4 and 5 proteins. (McKinsey et al., 2000a,b).

The inventor, in collaboration with others, has previously shown that the association between HDAC's and MEF2 is controlled by phosphorylation, and that protein kinases that were as then unidentified mediated the HDAC-MEF2 association (McKinsey et al., 2002). Mutant HDAC's lacking phosphorylation sites acted as signal-resistant repressors to cardiomyocyte hypertrophy and HDAC knock out mice were hypersensitive to heart failure and hypertrophy (Zhang et al., 2002). It has also has been shown that certain HDAC inhibitors are anti-hypertrophic. In other contexts, recent research has also highlighted the important role of HDACs in cancer biology. In fact, various inhibitors of HDACs are being tested for their ability to induce cellular differentiation and/or apoptosis in cancer cells. (Marks et al., 2000). Such inhibitors include suberoylanilide hydroxamic acid (SAHA) (Butler et al., 2000; Marks et al., 2001); m-carboxycinnamic acid bis-hydroxamide (Coffey et al., 2001); and pyroxamide (Butler et al., 2001).

All of the aforementioned findings demonstrate the important role of HDAC's in disease progression, and specific data demonstrates that the nuclear compartmentalization of HDACs is a key factor in cardiac disease. HDACs that are nuclear repress MEF2 dependent gene activation, and as such are anti-hypertrophic. Thus, finding a way to keep HDACs nuclear, or to find a way to inhibit export of HDACs from the nucleus, represents a potential therapeutic target in the treatment or prevention of hypertrophy or heart failure. This inhibition need not be directed at HDACs; generally inhibiting export of proteins from the nucleus will achieve the same results as targeting specific inhibitors of HDAC export. To date, there have been no reports of such strategies.

### SUMMARY OF THE INVENTION

Thus, in accordance with the present invention, there is provided a method of treating pathologic cardiac hypertrophy and heart failure comprising (a) identifying a patient having cardiac hypertrophy or heart failure; (b) selecting a known non-selective inhibitor of protein nuclear export and (c) administering said inhibitor to said patient. The inhibitor may be either reversible or irreversible. Administering may comprise intravenous, oral, transdermal, sustained release, suppository, or sublingual administration. The method may further comprise administering a second therapeutic regimen, such as a beta blocker, an iontrope, diuretic, ACE-I, AII antagonist, a Ca⁺⁺-blocker, and HDAC inhibitor, a TRP channel inhibitor, a 5-HT2 receptor agonist, or a 5-HT2 receptor antagonist. The second therapeutic regimen may be administered at the same time as the inhibitor of nuclear export, or either before or after the inhibitor of nuclear export. The treatment may improve one or more symptoms of heart failure cardiac failure such as providing increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, wall tension and wall thickness, quality of life, disease-related morbidity and mortality, decreased remodeling, ventricular dilation, or improving pump performance, decreasing necrosis, arrhythmia, fibrosis, energy starvation or apoptosis. In particular embodiments, the patient is a human.

In yet another embodiment, there is provided a method of preventing pathologic cardiac hypertrophy and heart failure comprising (a) identifying a patient at risk of developing cardiac hypertrophy or heart failure; (b) selecting a known non-selective inhibitor of protein nuclear export and (c) administering said inhibitor to said patient. Administration may comprise intravenous, oral, transdermal, sustained release, suppository, or sublingual administration. The patient at risk may exhibit one or more of long standing uncontrolled hypertension, uncorrected valvular disease, chronic angina and/or recent myocardial infarction. In particular embodiments, the patient is a human.

In accordance with the preceding embodiments, the inhibitor of nuclear export may be any molecule that inhibits a pathways, mechanism, or protein directly involved in the export of proteins from the nucleus of a cell. This includes proteins, peptides, peptide aptamers (for a review of this technology, see Kau & Silver, 2003; hereinafter incorporated by reference) DNA molecules (including antisense), RNA molecules (including RNAi and antisense) and small molecules. The small molecules include, but are not limited to, of peptide aptamers, leptomycin B, valtrate, callystatin A, polyketides, PKF050-638, STI571, staurosporine and staurosporine-related compounds.

In yet a further embodiment of the invention, there is provided a method for identifying inhibitors of nuclear export comprising first providing a potential inhibitor of nuclear export, then treating a cell with said potential inhibitor of nuclear export and a second stimulus, and then measuring the amount of class II HDAC that is exported from the nucleus in response to said second stimulus where the second stimulus is a stimulation that would lead to export of class II HDAC from the nucleus; wherein a decrease in the amount of class II HDAC that is exported from the nucleus in response to the stimulus, as compared to a cell not treated with said potential inhibitor of nuclear export, identifies said potential inhibitor as an inhibitor of nuclear export. The class II HDAC may be selected from the group consisting of HDAC 4, HDAC 5, HDAC 6, HDAC 7, HDAC9 and HDAC 10. The class II HDAC may be tagged with an agent that allows them to be microscopically observed, and that agent may be selected from GFP, RFP, or YFP. The cell may be a myocyte, a cardiomyocyte, and it may be a neonatal rat ventricular myocyte. The second stimulus may be phenylephrine, endothelin, fetal bovine serum, prostaglandin, PMA, or angiotensin. The HDAC's may also be epitope tagged and a labeled antibody may be used to detect the epitope.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Effect of a CRM-1 inhibitor on ANF secretion from cardiomyocytes. Primary neonatal rat ventricular myocytes (NRVM) were treated for 48 hrs with PE (20 mM) or FBS (5%) in the absence or presence of the indicated concentrations of leptomycin B (LMB). ELISA was employed to measure concentrations of secreted ANF in culture supernatants. The results are graphed as the means +/− standard deviations from eight independent samples.

FIG. 2—A CRM-1 inhibitor blocks induction of fetal cardiac gene mRNA transcripts. NRVM were treated for 48 hrs with PE (20 mM) in the absence or presence of LMB (18.5 nM). Total RNA was prepared and the indicated transcripts were detected by RNA dot blot analysis with radiolabeled oligonucleotide probes.

FIG. 3—A CRM-1 inhibitor blocks induction of b-MyHC protein expression. NRVM were cultured for 48 hrs with PE (20 mM) in the absence or presence of LMB (18.5 nM). Levels of β-MyHC protein were measured by cytoblot analysis and are graphed as percent expression relative to that found in untreated controls (set at 100%). Values represent means +/− standard deviations from eight independent samples.

FIG. 4—A CRM-1 inhibitor blocks agonist-mediated increases in cardiomyocyte protein synthesis. NRVM were treated with PE (20 mM) or FBS (5%) for 48 hours in the absence or presence of LMB (18.5 nM). Total cellular protein was quantified by Bradford assay and is depicted as percent of untreated cells. Values are averages from eight independent samples +/− standard deviation.

FIG. 5—A CRM-1 inhibitor blocks agonist-mediated increases in cardiomyocyte size. NRVM were treated with PE (20 mM) or FBS (5%) for 48 hours in the absence or presence of LMB (18.5 nM). NRVM were tryspinized and cell volumes determined by Coulter Counter analysis. Average cell volumes (mm3) were determined for 1×104 cells and are depicted as % of untreated controls.

FIG. 6—CRM-1 inhibition does not alter cardiomyocyte viability. Adenylate kinase (AK) was detected in culture medium of cardiomyocytes following 48 hours of stimulation with PE (20 mM) or FBS (5%) in the absence or presence of LMB (18.5 nM). Values represent the means +/− standard deviations from eight independent samples. AK levels did not increase in the medium in cells treated with LMB, indicating that LMB is not generally toxic to cardiomyocytes. The higher values from FBS-treated cells are a consequence of AK present in serum.

FIG. 7—Thyroid hormone-mediated changes in MyHC protein expression are unaffected by CRM-1 inhibition. NRVM were treated for 48 hours with T3 (3 nM) in the absence or presence of LMB (18.5 nM). Effects of LMB on T3-mediated regulation of α- and β-MyHC protein expression were assessed by cytoblot analysis. Values represent averages from at least 8 independent samples, +/− standard deviation.

FIG. 8—Thyroid hormone-mediated induction of SERCA2 protein expression is unaffected by CRM-1 inhibition. NRVM were treated for 48 hours with T3 (3 nM) or PE (20 mM) in the absence or presence of LMB (18.5 nM). Effects of LMB on SERCA2 protein expression were assessed by immunoblot analysis with anti-SERCA2 antibodies.

FIG. 9—A novel CRM-1 inhibitor blocks agonist-mediated ANF expression. NRVMs were treated for 48 hrs with PE (20 μM) in the absence or presence of the novel CRM-1 inhibitor 5219668 (1.25 μM). ELISA was employed to measure concentrations of secreted ANF in culture supernatants. The results are graphed as the means +/− standard deviations from eight independent samples.

FIG. 10—A novel CRM-1 inhibitor blocks agonist-mediated increases in cardiomyocte protein synthesis. NRVMs were treated with PE (20 μM) or FBS (5%) for 48 hours in the absence or presence of 5219668 (1.25 μM). Total cellular protein was quantified by Bradford assay and is depicted as percent of untreated cells. Values are averages from eight independent samples +/− standard deviation.

FIG. 11—A novel CRM-1 inhibitor blocks agonist-mediated increases in cardiomyocyte size. NRVMs were treated with PE (20 μM) for 48 hours in the absence or presence of 5219668 (1.25 μM). NRVMs were tryspinized and cell volumes determined by Coulter Counter analysis. Average cell volumes (mm3) were determined for 1×104 cells and are depicted as % of untreated controls.

### DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are usually those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

In light of the limitations of the current therapies, the inventors propose a novel mechanism to regulate the gene cascade required for the development of hypertrophy and heart failure. The inventor and others have previously shown a major role for MEF2 as a transcription factor required for the activation of “fetal genes” which are turned on aberrantly when the heart responds to insult or injury (see U.S. Pat. No. 6,372,957 for a review, hereinafter incorporated by reference). The inventor, in collaboration, has also shown a role for HDACs in modulating MEF2 dependent gene transcription, and further shown that when class II HDACs in particular are exported from the nucleus, MEF2 dependent gene transcription begins (for a review see U.S. Patent Application 2003/144340, hereinafter incorporated by reference). The inventor now reports that non-selective inhibition of export of proteins from the nucleus prevents or reverses the genetic and molecular cascades that lead to hypertrophy and heart failure. Surprisingly, such inhibition is not toxic to cells. Thus, and in accordance with the present invention, the inventor describes novel therapeutic methods for treating cardiac hypertrophy and heart failure by inhibiting export of proteins from the nucleus.

I. Transcriptional Pathway for Heart Failure or Cardiac Hypertrophy

The inventor and others have previously shown that a variety of genes are involved in the pathways that are activated when the heart is damaged or stressed. The individual components of these pathways as they relate to heart failure or cardiac hypertrophy are discussed in further detail herein below.

A. Calcineurin

It is known that Ca(++) activation is involved in a variety of forms of heart failure and heart disease. Ca(++) store depletion, or a raise in the cytoplasmic Ca(++) levels in the cell, has been show to stimulate a calcineurin dependent pathway for cardiac hypertrophy. Calcineurin is a ubiquitously expressed serine/threonine phosphatase that exists as a heterodimer, comprised of a 59 kD calmodulin-binding catalytic A subunit and a 19 kD Ca(++)-binding regulatory B subunit (Stemmer and Klee, 1994; Su et al., 1995). Calcineurin is uniquely suited to mediate the prolonged hypertrophic response of a cardiomyocyte to Ca(++) signaling because the enzyme is activated by a sustained Ca(++) plateau and is insensitive to transient Ca(++) fluxes as occur in response to cardiomyocytc contraction (Dolmetsch et al., 1997).

Activation of calcineurin is mediated by binding of Ca(++) and calmodulin to the regulatory and catalytic subunits, respectively. Previous studies showed that over-expression of calmodulin in the heart also results in hypertrophy, but the mechanism involved was not determined (Gruver et al., 1993). It is now clear that calmodulin acts through the calcineurin pathway to induce the hypertrophic response. Calcineurin has been shown previously by the inventors to phosphorylate NF-AT3, which subsequently acts on the transcription factor MEF-2 (Olson et al., 2000). Once this event occurs, MEF-2 activates a variety of genes known as fetal genes, the activation of which inevitably results in hypertrophy and heart failure.

CsA and FK-506 bind the immunophilins cyclophilin and FK-506-binding protein (FKBP12), respectively, forming complexes that bind the calcineurin catalytic subunit and inhibit its activity. CsA and FK-506 block the ability of cultured cardiomyocytes to undergo hypertrophy in response to AngII and PE (both strong agonists of hypertrophy in vitro and in vivo). Both of these hypertrophic agonists have been shown to act by elevating intracellular Ca(++), which results in activation of the PKC and MAP kinase signaling pathways (Sadoshima et al., 1993; Sadoshima and Izumo, 1993; Kudoh et al., 1997; Yamazaki et al., 1997, Zou et al., 1996). CsA does not interfere with early signaling events at the cell membrane, such as PI turnover, Ca(++) mobilization, or PKC activation (Emmel et al., 1989). Thus, its ability to abrogate the hypertrophic responses of AngII and PE suggests that calcineurin activation is an essential step in the AngII and PE signal transduction pathways.

B. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Re1 homology domain (RHD) (Rooney et al., 1994; Hoey et al., 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

NF-AT3 is a 902-amino acid with a regulatory domain at its amino-terminus that mediates nuclear translocation and the Re1-homology domain near its carboxyl-terminus that mediates DNA binding. There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. CsA or FK506 may block each step of the activation pathway. This implies, and the inventors' earlier studies have shown, that calcineurin is the protein responsible for NF-AT activation.

Thus, in T cells, many of the changes in gene expression in response to calcineurin activation are mediated by members of the NF-AT family of transcription factors, which translocate to the nucleus following dephosphorylation by calcineurin. Many observations support the conclusion that NF-AT also is an important mediator of cardiac hypertrophy in response to calcineurin activation. NF-AT activity is induced by treatment of cardiomyocytes with AngII and PE. This induction is blocked by CsA and FK-506, indicating that the induction is calcineurin-dependent. NF-AT3 synergizes with GATA4 to activate the cardiac specific BNP promoter in cardiomyocytes. Also, expression of activated NF-AT3 in the heart is sufficient to bypass all upstream elements in the hypertrophic signaling pathway and evoke a hypertrophic response.

Prior work has demonstrated that the C-terminal portion of the Re1-homology domain of NF-AT3 interacts with the second zinc finger of GATA4, as well as with GATA5 and GATA6, which are also expressed in the heart. The crystal structure of the DNA binding region of NF-ATc has revealed that the C-terminal portion of the Re1-homology domain projects away from the DNA binding site and also mediates interaction with AP-1 in immune cells (Wolfe et al., 1997).

According to a model previously proposed by the inventors, hypertrophic stimuli such as AngII and PE, which lead to an elevation of intracellular Ca(++), result in activation of calcineurin. NF-AT3 within the cytoplasm is dephosphorylated by calcineurin, enabling it to translocate to the nucleus where it can interact with GATA4, and then activate the transcription factor MEF-2, a family of transcription factors that are normally repressed by a tight association with class II HDAC's.

Results of previous work by the inventors have shown that calcineurin activation of NF-AT3 regulates hypertrophy in response to a variety of pathologic stimuli and suggests a sensing mechanism for altered sarcomeric function. Of note, there are several familial hypertrophic cardiomyopathies (FHC) caused by mutations in contractile protein genes, which result in subtle disorganization in the fine crystalline-like structure of the sarcomere (Watkins et al., 1995; Vikstrom and Leinwand, 1996). It is unknown how sarcomeric disorganization is sensed by the cardiomyocyte, but it is apparent that this leads to altered Ca(++) handling (Palmiter and Solaro, 1997; Botinelli et al., 1997; Lin et al., 1996). Calcineurin, as discussed above, is one of the sensing molecules that couples altered Ca(++) handling associated with FHC with cardiac hypertrophy and heart failure.

C. MCIP

Another gene that is associated with heart failure and hypertrophy, primarily due to its tight association with and regulation by Calcineurin, is the human gene (DSCR1) encoding MCIP1, one of 50-100 genes that reside within a critical region of chromosome 21 (Fuentes et al., 1997; Fuentes et al., 1995), trisomy of which gives rise to the complex developmental abnormalities of Down syndrome, which include cardiac abnormalities and skeletal muscle hypotonia as prominent features (Epstein, 1995). ZAKI-4 was identified from a human fibroblast cell line in a screen for genes that are transcriptionally activated in response to thyroid hormone (Miyazaki et al., 1996).

MCIP1 directly binds and inhibits calcineurin, functioning as an endogenous feedback inhibitor of calcineurin activity. Overexpression of MCIP1 in the hearts of transgenic animals is anti-hypertrophic; MCIP1 attenuates in vivo models of both calcineurin-dependent hypertrophy (Rothermel et al., 2001) and pressure-overload-induced hypertrophy (Hill et al., 2002). MCIP1 also acts as a substrate for phosphoryalation by MAPK and GSK-3, and calcineurin's phosphatase activity. Residues 81-177 of MCIP1 retain the calcineurin inhibitory action.

Binding of MCIP1 to calcineurin does not require calmodulin, nor does MCIP interfere with calmodulin binding to calcineurin. This suggests that the surface of calcineurin to which MCIP1 bindings does not include the calmodulin binding domain. In contrast, the interaction of MCIP1 with calcineurin is disrupted by FK506:FKBP or cyclosporin:cyclophylin, indicating that the surface of calcineurin to which MCIP1 binds overlaps with that required for the activity of immunosuppressive drugs.

D. MEF2

As mentioned above, NF-AT3 activation by Calcineurin leads to the activation of another family of transcription factors, the monocyte enhancer factor-2 family (MEF2), which are known to play an important role in morphogenesis and myogenesis of skeletal, cardiac, and smooth muscle cells (Olson et al., 1995). MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences (Brand, 1997; Olson et al., 1995). These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF2 factors regulate myogenesis through combinatorial interactions with other transcription factors.

Loss-of-function studies indicate that MEF2 factors are essential for activation of muscle gene expression during embryogenesis. The expression and functions of MEF2 proteins are subject to multiple forms of positive and negative regulation, serving to fine-tune the diverse transcriptional circuits in which the MEF2 factors participate. MEF-2 is bound in an inactive form in the healthy heart by class II HDACS (see supra), and when MEF-2 is activated it is released from the HDAC and activates the fetal gene program that is so deleterious for the heart.

E. Histone Deacetylase

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Eleven different HDACs have been cloned from vertebrate organisms. The first three human HDACs identified were HDAC 1, HDAC 2 and HDAC 3 (termed class I human HDACs), and HDAC 8 (Van den Wyngaert et al., 2000) has been added to this list. Recently class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Additionally, HDAC 11 has been identified but not yet classified as either class I or class II (Gao et al., 2002). All share homology in the catalytic region. HDACs 4, 5, 7, 9 and 10 however, have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDAC's repress MEF2 activity is not completely understood, but it is known that HDACs must remain in the nucleus to be bound to MEF2 and repress MEF2 dependent gene activation. One possibility is that HDAC binding to MEF2 inhibits MEF2 transcriptional activity, either competitively or by destabilizing the native, transcriptionally active MEF2 conformation. It also is possible that class II HDAC's require dimerization with MEF2 to localize or position HDAC in a proximity to histones for deacetylation to proceed.

HDACs can be inhibited through a variety of different mechanisms—proteins, peptides, and nucleic acids (including antisense, RNAi molecules, and ribozymes). Methods are widely known to those of skill in the art for the cloning, transfer and expression of genetic constructs, which include viral and non-viral vectors, and liposomes. Viral vectors include adenovirus, adeno-associated virus, retrovirus, vaccina virus and herpesvirus. Also known are small molecule HDAC inhibitors. Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al., 1996). It is commercially available from a variety of sources including BIOMOL Research Labs, Inc., Plymouth Meeting, Pa.

A variety of inhibitors for histone deacetylase have been identified. The proposed uses range widely, but primarily focus on cancer therapy. (See Saunders et al., 1999; Jung et al, 1997; Jung et al. 1999; Vigushin et al., 1999; Kim et al., 1999; Kitazomo et al., 2001; Vigusin et al., 2001; Hoffmann et al., 2001; Kramer et al., 2001; Massa et al., 2001; Komatsu et al., 2001; Han et al., 2000). Such therapy is the subject of NIH sponsored clinical trials for solid and hematological tumors. HDAC's also increase transcription of transgenes, thus constituting a possible adjunct to gene therapy. (Yamano et al., 2000; Su et al., 2000). Additionally, the following references describe histone deacetylase inhibitors which may be selected for use in the current invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

II. Nuclear Export

All the genes mentioned in the preceding section present enticing therapeutic targets for heart failure and hypertrophy. By inhibiting any part of the cascades or pathways that recruit these proteins, one may hope to impede cardiac hypertrophy and subsquent failure of the heart muscle. One method or mode of inhibiting these cascades would be to prevent the final stage of the cascades, gene transcription, from being activated. As such, a way to interfere with the activation of gene expression, or for controlling the related aberrant gene expression, would be a novel and potentially powerful tool for treating or preventing hypertrophy or heart failure.

A common mechanism for controlling gene expression involves altering the subcellular distribution of transcriptional regulators. A multitude of transcription factors and co-factors possess nuclear localization sequences (NLSs) and nuclear export signals (NESs) that mediate entry into and exit from the nucleus, respectively. Frequently, signal transduction pathways that impinge on transcriptional regulators function by positively or negatively affecting the activity of these intrinsic targeting domains.

For proteins over ˜40 kilodaltons (kDa), passage into and out of the nucleus is governed by the nuclear pore complex (NPC), a mega-kDa complex embedded in the nuclear envelope (reviewed in Jans et al., 2000). Positively charged NLSs are bound by importin α and β, which tether cargo to the cytosolic face of the NPC and facilitate translocation of proteins into the nucleus. The CRM-1 protein, also referred to as exportin, mediates transit of proteins out of the nucleus (Fornerod et al., 1997), although CRM-1-independent mechanisms for nuclear export exist (Holaska et al., 2001). CRM-1 binds hydrophobic NESs together with the small GTP binding protein Ran, and these ternary complexes are shuttled out of the nucleus through a series of interactions with the NPC. The capacity of nuclear import/export machinery to access NLS or NES is often dictated by signaling events that culminate in exposure or masking of these regulatory sequences (reviewed in Cyert, 2001). This may occur through direct modification of the target protein or via modification of an associated factor. Phosphorylation has been most commonly implicated in this mode of control, although roles for other types of post-translational modifications (e.g., acetylation) in the regulation of protein localization have recently been revealed (Chen et al., 2001).

Involvement of CRM-1 in the regulation of specific proteins can be assessed employing leptomycin B (LMB), a streptomyces metabolite. LMB covalently attaches to cysteine-539 of CRM-1, thereby prohibiting the export factor from associating with NES-containing cargo (Kudo et al., 1999). Recently, a synthetic small molecule, PKF050-638, was identified as an inhibitor of CRM-1 function by virtue of its ability to block nuclear export of HIV Rev protein (Daelemans et al., 2002). Additionally, valtrate, purified from roots of the medicinal plant, Valerianae radix, was shown to function as a CRM-1 antagonist (Murakami et al., 2002). Like LMB, both PKF050-638 and valtrate inhibit CRM-1 by covalent modification of cysteine-539.

In light of the fact that HDACs must be exported from the nucleus in order to release MEF2, and thus activate the aforementioned aberrant gene program that leads to hypertrophy, the inventor investigated whether non-selective inhibition of nuclear export could inhibit hypertrophy. Such inhibition would, whether specifically or indirectly, inhibit export of class II HDACs from the nucleus, keeping them tightly bound to MEF2 and allowing them to act in a repressive manner. Treatment of heart failure or hypertrophy by inhibiting MEF2 activation in this manner, thereby preventing the HDAC-MEF2 dissociation from occurring, would represent a major leap forward over the current methods available for treating patients suffering from these diseases. As such, and in accordance with the present invention, the inventors disclose that non-specific inhibition of nuclear export is indeed anti-hypertrophic, non-toxic, and an efficacious method of therapy for heart failure or hypertrophy. Thus, one of skill in the art will treat patients with a drug specifically selected for its ability to provide non-selective inhibition of nuclear protein export.

III. Methods of Treating Heart Failure and Cardiac Hypertrophy

A. Current Therapeutic Regimens for Heart Failure and Hypertrophy

Heart failure of some forms may curable and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy. Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review, see De Feo et al., 2003; DiBianco, 2003). Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty (Dumcius et al., 2003) This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn & Bristow, 1996). Other pharmaceutical agents that have been disclosed for treatment of cardiac hypertrophy include angiotensin II receptor antagonists (U.S. Pat. No. 5,604,251) and neuropeptide Y antagonists (PCT Publication No. WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

As can be seen from the discussion above, there is a great need for a successful treatment approach to heart failure and hypertrophy. In one embodiment of the present invention, methods for the treatment of cardiac hypertrophy or heart failure utilizing inhibitors of nuclear export are provided. For the purposes of the present application, treatment comprises reducing one or more of the symptoms of heart failure or cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of nuclear export may prevent heart failure or cardiac hypertrophy and their associated symptoms from arising.

B. Inhibiting Nuclear Export

1. Pharmaceutical Inhibition

Inhibition of nuclear export is a fairly recent focus of research, and as such only a few such inhibitors have been characterized. “Non-selective inhibition of nuclear export” is define as the inhibition of any one of multiple pathways, mechanisms, or proteins directly involved in the export of proteins from the nucleus of a cell. Such inhibition does not involve modifications to the proteins being exported themselves that thereby makes them unavailable to the export machinery. The short list of identified compounds known to non-selectively inhibit nuclear export contains leptomycin B, PKF050-638, STI571, staurosporine and staurosporine-related compounds (Kau and Silver, 2003). However, new inhibitors continue to be developed and reported in the literature.

2. Antisense Constructs

An alternative approach to inhibiting nuclear export is to target the genes required for nuclear export and block them via antisense technologies. Antisense methodology takes advantage of the fact that nucleic acids tend to pair with “complementary” sequences. By complementary, it is meant that polynucleotides which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences that are completely complementary will be sequences that are entirely complementary throughout their entire length and have no base mismatches. Other sequences with lower degrees of homology also are contemplated. For example, an antisense construct that has limited regions of high homology, but also contains a non-homologous region (e.g., ribozyme; see below), could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

3. Ribozymes

Another general class of inhibitors that could be used to therapeutically target export specific genes are ribozymes. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

4. RNAi

RNA interference (also referred to as “RNA-mediated interference” or RNAi) is another mechanism by which nuclear export mechanisms may be inhibited. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This result was demonstrated by Elbashir et al. (2001), wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

5. Antibodies

In certain aspects of the invention, antibodies may find use as inhibitors of nuclear export machinery. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

6. Peptide Aptamers

Peptide aptamers represent yet another potential mechanism for either inhibiting nuclear export or disturbing/treating a secondary signaling cascade related to or involved in cardiac hypertrophy or heart failure. Recently, the ability to manipulate individual genes has driven the development of reverse genetics, in which the function of genes is inferred from the phenotypes that arise from their mutation. In diploids, reverse genetics also typically requires generation of homozygotes in the mutated gene. To circumvent this requirement, a number of dominant “reverse genetic” methods to inactivate gene function have been devised, including inhibition by drugs, expression of dominant-negative proteins, injection of antibodies, expression of antisense RNAs, expression of nucleic acid aptamers, and expression of peptide aptamers (Geyer et al., 1999).

The ability to specifically interfere with the function of proteins of pathological significance has been a goal for molecular medicine for many years. Peptide aptamers are proteins that contain a conformationally constrained peptide region of variable sequence displayed from a scaffold (Geyer et al., 1999). Peptide aptamers comprise a new class of molecules, with a peptide moiety of randomized sequence, which are selected for their ability to bind to a given target protein under intracellular conditions (Hoppe-Seyler et al., 2004). They have the potential to inhibit the biochemical activities of a target protein, can delineate the interactions of the target protein in regulatory networks, and identify novel therapeutic targets. Peptide aptamers represent a new basis for drug design and protein therapy, with implications for basic and applied research, for a broad variety of different types of diseases (Hoppe-Seyler et al., 2004).

Peptide aptamers from combinatorial libraries can be dominant inhibitors of gene function. Researchers have used two-hybrid systems to select aptamers based on Escherichia coli thioredoxin (TrxA) that recognize specific proteins and allelic variants. Apterms have been selected against Cdk2 (Colas et al., 1996), Ras (Xu et al, 1997), E2F (Fabbrizio et al., 1999), and HIV-1 Rev (Cohen et al., 1998). Apterms have been used in mammalian cells (Cohen et al, 1998) and in Drosophila melanogaster (Kolonin et al., 1998). These recent results demonstrate the power and potential utility of peptide aptamers, both as stand-alone therapeutics and even as a potential class of inhibitors of nuclear export. As such they could be used to block nuclear export, or they could be used in conjunction with an inhibitor of nuclear export as a dual or combination therapy.

C. Combined Therapy

Treatment regimens may vary depending on the clinical situation. However, long term maintenance would appear to be appropriate in most circumstances when dealing with either heart failure or hypertrophy. It also may be desirable treat hypertrophy or heart failure with intermittent regiments, such as within brief windows during disease progression.

In certain embodiments of the invention, it is envisioned to use an inhibitor of nuclear export in combination with other therapeutic modalities. Thus, in addition to the therapies described above, one may also provide to the patient as a combined therapy more “standard” pharmaceutical cardiac therapies. Examples of therapies (including those already mentioned) include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, HDAC inhibitors, TRP channel inhibitors, and 5-HT2 receptors modulators.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using an inhibitor of nuclear export may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor of nuclear export, or the other agent will be desired. In this regard, various combinations may be employed. By way of illustration, where the inhibitor of nuclear export is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

D. Adjunct Therapeutic Agents (Pharmaceutical)

Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutblol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators such as phosphodiesterase inhibitors or endothelin receptor antagonists, and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include ambristentan, amotriphene, bendazol, benfurodil hemisuccinate, benziodarone, bosentan, chloracizine, chromonar, clobenfurol, clonitrate, darusentan, dilazep, dipyridamole, droprenilamine, efloxate, enoximone, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, milrinone, nicorglycerin, pentaerythritol tetranitrate, pentrinitrol, perhexiline, pimethylline, sitaxsentan, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quantemary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

9. TRP Channel Inhibitors

Additional insights into the possible origin of the hypertrophic calcium signal have come from studies of the calcineurin-NFAT pathway in the immune system (Crabtree and Olson, 2002). During lymphocyte activation, ligand binding to T-cell receptors stimulates PLC activation and the production of IP3, which induces a transient release of calcium from intracellular stores via the IP3 receptor (the predominant calcium release channel in lymphocytes). This transient calcium release, however, is insufficient to activate calcineurin and subsequent NFAT-dependent responses. Rather, the initial calcium release from intracellular stores triggers a secondary influx of extracellular calcium through specialized Calcium Release Activated Calcium (CRAC) channels. It is this influx of extracellular calcium that produces the sustained calcium signal capable of activating the calcineurin pathway. Given the degree to which the calcineurin-NFAT signaling module is utilized in a variety of cell types, it is reasonable to predict that a similar mechanism (e.g., a cardiac CRAC channel) may be responsible for activation of this pro-hypertrophic pathway in the heart.

While the electrophysiologic characteristics of cardiac CRAC channels have been extensively studied, the specific genes encoding these channels have yet to be completely identified. Nonetheless, the genes responsible for cardiac CRAC channel characteristics represent a starting point for the cascade leading to hypertrophy and are potential therapeutic targets for both heart failure and hypertrophy, their genetic identity remains obscure. The channel protein CaT1 was recently demonstrated to possess the expected electrophysiologic properties of a CRAC channel (Yue et al., 2001). CaT1 is a member of a large group (approximately 20 genes) of non-voltage-gated plasma membrane cation channels collectively known as the Transient Receptor Potential (TRP) family (Venneken et al., 2002). The TRP family can be divided into three subfamilies on the basis of sequence homology: the TRPC (canonical) subfamily, the TRPV (vanilloid) subfamily and the TRPM (melastatin) subfamily. TRP family members clearly function as calcium influx channels in a variety of tissues, but relatively little is currently known about the specific physiological roles and modes of regulation of this emerging ion channel family.

Members of the TRPC subfamily are known effectors of G-protein coupled receptors, and are directly activated by diacylglycerol and IP3 produced as a result of GPCR-dependent PLC activation. TRPC subfamily members also function as CRAC channels; they are activated in response to depletion of intracellular calcium stores. The specific mechanism coupling store depletion to calcium influx is unknown, but in the case of TRPC3, the channel is thought to interact directly with the IP3 receptor. Interestingly, expression level of the TRPC3 channel has been shown to influence how the channel is regulated; PLC activation is the predominant regulatory mode at high levels of channel expression, while lower expression levels favor store depletion (Vasquez et al., 2003). Crucially, TRPC channels have recently been demonstrated to contribute to pathologic calcium signaling in muscle (Vandebrouck et al., 2002). Skeletal muscle fibers from patients suffering from Duchenne muscular dystrophy exhibit abnormally increased calcium influx, which contributes to the dystrophic phenotype via activation of calcium-dependent proteases. Antisense repression of TRPC expression in dystrophic muscle fibers reduced the abnormal calcium influx, confirming the role of this channel in the disease process.

Other TRP subfamily members are less well studied, but appear to respond to different stimuli. In addition to regulation by store depletion, TRPV channels are also activated by mechanical stretch, heat and the hot pepper compound capsaicin. In contrast, TRPM channels are activated by cold temperatures and compounds like menthol.

The TRP channels are associated with all of the cascades that lead to heart failure and hypertrophy as a starting point, a therapeutic bottleneck, for inhibiting the transcriptional and translational pathways associated with heart failure and hypertrophy. As such, inhibiting these channels represents yet another alternative treatment to heart failure and hypertrophy.

10. 5-HT2 Receptor Modulators

Another recently evolving line of treatment for heart disease entails modulation of the serotonin receptors (5-HT2) on the heart. Cardiac G-protein coupled receptor signaling pathways may feed into the calcium-dependent hypertrophic signaling module by a variety of mechanisms. Signaling via one prominent class of G-protein coupled receptors, the 5-HT2 receptors, activates phospholipase C in a variety of cell types. Activated phospholipase C produces IP3 and diacylglycerol, second messengers which cause concentrations of intracellular calcium to rise. Stimulation of 5-HT2 receptors thus activates the calcineurin signaling module (Day et al., 2002). Consistent with this observation, an endogenous calcineurin inhibitory protein of the MCIP family has been shown to attenuate serotonergic signaling (Lee et al., 2003). Cardiac serotonergic signaling may also interface with other pro-hypertrophic signaling modules; serotonin has been shown to activate S6 kinase (Khan et al., 2001), a key regulator of translation during myocyte hypertrophy.

It has been shown that there is a set of membrane bound G-protein coupled receptors, previously described in the art as serotonin receptors, which are involved in the cellular cascades that lead to heart damage, and subsequently heart failure, hypertrophy, and PPH. These receptors, the 5-HT2 serotonin receptors, like the TRP Channels, are a starting point for a number of important signaling pathways already known to be important in the cellular cascade towards hypertrophy. Thus, modulation of these receptors represents yet another therapeutic method for treating cardiac hypertrophy, PPH, and heart failure.

Within the 5-HT (2) family, 5-HT (2A), 5-HT (2B) and 5-HT (2C) subtypes are known to exist. These subtypes share sequence homology and display similarities in their specificity for a wide range of ligands. The 5-HT (2B) receptor, initially termed 5-HT (2F), or serotonin-like receptor, was first characterized in rat isolated stomach fundus (Clineschmidt et al., 1985; Cohen and Wittenauer, 1987) and initially cloned from rat (Foguet et al., 1992) followed by the cloning of the human 5-HT (2B) receptor (Schmuck et al., 1994; Kursar et al., 1994). The 5-HT (2C) receptor, widely distributed in the human brain, was first characterized as a 5-HT (1C) subtype (Pazos et al., 1984) and was subsequently recognized as belonging to the 5-HT (2) receptor family (Pritchett et al., 1988).

Because of the similarities in the pharmacology of ligand interactions at 5-HT (2B) and 5-HT (2C) receptors, many of the therapeutic targets that have been proposed for 5-HT (2C) receptor antagonists are also targets for 5-HT (2B) receptor antagonists. Current evidence strongly supports a therapeutic role for 5-HT (2B/2C) receptor antagonists in treating anxiety (e.g., generalized anxiety disorder, panic disorder and obsessive compulsive disorder), alcoholism and addiction to other drugs of abuse, depression, migraine, sleep disorders, feeding disorders (e.g., anorexia nervosa) and priapism. Additionally, current evidence strongly supports a therapeutic role for selective 5-HT (2B) receptor antagonists that will offer distinct therapeutic advantages collectively in efficacy, rapidity of onset and absence of side effects. Such agents are expected to be useful in the treatment of hypertension, disorders of the gastrointestinal tract (e.g., irritable bowel syndrome, hypertonic lower esophageal sphinter, motility disorders), restenosis, asthma and obstructive airway disease, and prostate hyperplasia (e.g., benign prostate hyperplasia).

Recent research has highlighted the potential imporantance of these receptors in cardiovascular diseases, specifically in relation to elevated 5-HT (serotonin) levels, but the diversity of 5-HT receptors and the lack of 5-HT receptor isotype-specific pharmacological agents have complicated attempts to make any significant clinical advances in this area (Nebigil and Maroteaux, 2003). Nebigil et al. have found that there is a significant role for serotnonin in the heart, and that knocking out the 5-HT2b receptor can inhibit apoptosis and modulate heart disease, and that this modulation may occur through the PI3-Kinase pathway (Negibil et al., 2003b). Negibil and others have also showed that the 5-HT2b receptor is needed for proper development of the heart, but overexpression of the same receptor can lead to abnormal mitochondrial function and cardiac hypertrophy, and that 5-HT2b receptors are upregulated in the pulmonary arteries of patients suffering from PPH (Negibil et al., 2000; Negibil et al., 2003c; Launay et al., 2002). These results underscore the potential utility in modulation of this receptor subtype for the treatment of a variety of cardiovascular diseases, and exemplify how such modulation could be beneficial when used in conjunction with an inhibitor of nuclear export.

E. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

F. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. Nos. 4,256,108, 4,166,452, and 4,265,874, to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soybean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents that have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient that is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The previously mentioned formulations are all contemplated for treating patients suffering from heart failure or hypertrophy.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

IV. Screening Methods

The present invention further comprises methods for identifying inhibitors of nuclear export in cells that are useful in the prevention or treatment or reversal of cardiac hypertrophy or heart failure. These assays may comprise random screening of large libraries of candidate substances; alternatively, the assays may be used to focus on particular classes of compounds selected with an eye towards structural attributes that are believed to make them more likely to inhibit nuclear export or, more specifically, to inhibit the nuclear export of HDACs.

To identify an inhibitor of nuclear export, one generally will determine the nuclear localization of a class II HDAC in the presence and absence of the candidate substance. For example, a method generally comprises:
(a) providing a cardiomyocyte;
(b) contacting said cardiomyocyte with a candidate inhibitor substance and a hypertrophic stimulus; and
(c) measuring the amount of one or more class II HDACs remaining in the nucleus after exposure to the candidate inhibitory substance as compared to a cardiomyocyte not treated with the candidate inhibitor;
   wherein a decrease in the amount of HDAC exported out of the nucleus, as compared to the amount of nuclear localized HDAC in an untreated cell, identifies the candidate substance as an inhibitor of cardiac nuclear export.

Assays also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule or compound that may potentially inhibit the export of proteins from the nucleus of a cell. The candidate substance may be a protein or fragment thereof, a peptide aptamer, a small molecule, or even a nucleic acid. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to bind and inhibit nuclear export.

C. In Cyto Assays

The present invention also contemplates the screening of compounds in cells for their ability to inhibit nuclear export. Various cell lines can be utilized for such screening assays, including cells specifically engineered for this purpose.

D. In Vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria. Also, measuring toxicity and dose response can be performed in animals in a more meaningful fashion than in in vitro or in cyto assays.

V. Vectors for Cloning, Gene Transfer and Expression

Within certain embodiments, expression vectors are employed to express various products including HDACs, antisense molecules, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native promoter will be employed to drive expression of either the corresponding gene, a heterologous gene, a screenable or selectable marker gene, or any other gene of interest (i.e., HDACs, MEF2).

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., |
| | 1983; Grosschedl et al., 1985; Atchinson |
| | et al., 1986, 1987; Imler et al., 1987; |
| | Weinberger et al., 1984; Kiledjian et |
| | al., 1988; Porton et al.; 1990 |
| Immunoglobulin Light Chain | Queen et al., 1983; Picard et al., 1984 |
| T-Cell Receptor | Luria et al., 1987; Winoto et al., 1989; |
| | Redondo et al., 1990 |
| HLA DQ a and/or DQ β | Sullivan et al., 1987 |
| β-Interferon | Goodbourn et al., 1986; Fujita et al., |
| | 1987; Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al., 1989 |
| Muscle Creatine Kinase | Jaynes et al., 1988; Horlick et al., |
| (MCK) | 1989; Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., |
| | 1989 |
| Collagenase | Pinkert et al., 1987; Angel et al., |
| | 1987a |
| Albumin | Pinkert et al., 1987; Tronche et al., |
| | 1989, 1990 |
| α-Fetoprotein | Godbout et al., 1988; Campere et al., |
| | 1989 |
| t-Globin | Bodine et al., 1987; Perez-Stable et |
| | al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., |
| | 1981; Sleigh et al., 1985; Firak et al., |
| | 1986; Herr et al., 1986; Imbra et al., |
| | 1986; Kadesch et al., 1986; Wang et al., |
| | 1986; Ondek et al., 1987; Kuhl et al., |
| | 1987; Schaffner et al., 1988 |
| Polyoma | Swartzendruber et al., 1975; Vasseur et |
| | al., 1980; Katinka et al., 1980, 1981; |
| | Tyndell et al., 1981; Dandolo et al., |
| | 1983; de Villiers et al., 1984; Hen et |
| | al., 1986; Satake et al., 1988; Campbell |
| | and/or Villarreal, 1988 |
| Retroviruses | Kriegler et al., 1982, 1983; Levinson et |
| | al., 1982; Kriegler et al., 1983, 1984a, |
| | b, 1988; Bosze et al., 1986; Miksicek et |
| | al., 1986; Celander et al., 1987; |
| | Thiesen et al., 1988; Celander et al., |
| | 1988; Choi et al., 1988; Reisman et al., |
| | 1989 |
| Papilloma Virus | Campo et al., 1983; Lusky et al., 1983; |
| | Spandidos and/or Wilkie, 1983; Spalholz |
| | et al., 1985; Lusky et al., 1986; Cripe |
| | et al., 1987; Gloss et al., 1987; |
| | Hirochika et al., 1987; Stephens et al., |
| | 1987 |
| Hepatitis B Virus | Bulla et al., 1986; Jameel et al., 1986; |
| | Shaul et al., 1987; Spandau et al., |
| | 1988; Vannice et al., 1988 |
| Human Immunodeficiency | Muesing et al., 1987; Hauber et al., |
| Virus | 1988; Jakobovits et al., 1988; Feng et |
| | al., 1988; Takebe et al., 1988; Rosen et |
| | al., 1988; Berkhout et al., 1989; Laspia |
| | et al., 1989; Sharp et al., 1989; |
| | Braddock et al., 1989 |
| Cytomegalovirus (CMV) | Weber et al., 1984; Boshart et al., |
| | 1985; Foecking et al., 1986 |
| Gibbon Ape Leukemia Virus | Holbrook et al., 1987; Quinn et al., |
| | 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; |
| | | Stuart et al., 1985; |
| | | Imagawa et al., 1987, |
| | | Karin et al., 1987; |
| | | Angel et al., 1987b; |
| | | McNeall et al.,1989 |
| MMTV (mouse mammary | Glucocorticoids | Huang et al., 1981; |
| tumor virus) | | Lee et al., 1981; |
| | | Majors et al., 1983; |
| | | Chandler et al., 1983; |
| | | Ponta et al., 1985; |
| | | Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle Disease | |
| | Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, |
| | Antigen | 1990a, 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al, 1994; Kelly et al., 1995), the alpha actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the alpha7 integrin promoter (Ziober & Kramer, 1996), the brain natriuretic peptide promoter (LaPointe et al., 1995) and the alpha B-crystallin/small heat shock protein promoter (Gopal-Srivastava, R., 1995), alpha myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated, as an element of the expression cassette, is a terminator. These elements can serve to enhance message levels and to minimize read through from the cassette into other sequences.

B. Selectable Markers

In certain embodiments of the invention, the cells contain nucleic acid constructs of the present invention. A cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well as an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3, or both regions (Graham and Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. (Dubensky et al., 1984) successfully injected polyomavirus DNA in the form of calcium phosphate precipitates into liver and spleen of adult and newborn mice demonstrating active viral replication and acute infection. (Benvenisty and Neshif, 1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention there are contemplated means for transferring a naked DNA expression construct into cells that may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs, which can be employed to deliver a nucleic acid encoding a particular gene into cells, are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VI. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure or hypertrophy. “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In animal models, the response of treated transgenic animals and untreated transgenic animals may be compared using any of the assays described herein (in addition, treated and untreated non-transgenic animals may be included as controls). A compound that causes an improvement in any parameter associated with heart failure used in the screening methods of the instant invention may thereby be identified as a therapeutic compound.

The terms “compound” and “chemical agent” refer to any chemical entity, pharmaceutical, drug, and the like that can be used to treat or prevent a disease, illness, sickness, or disorder of bodily function. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the terms “antagonist” and “inhibitor” refer to molecules, compounds, or nucleic acids which inhibit the action of a cellular factor that may be involved in heart failure or cardiac hypertrophy. Antagonists may or may not be homologous to natural compounds in respect to conformation, charge or other characteristics. Thus, antagonists may be recognized by the same or different receptors that are recognized by an agonist. Antagonists may have allosteric effects that prevent the action of an agonist. Alternatively, antagonists may prevent the function of the agonist. In contrast to the agonists, antagonistic compounds do not result in pathologic and/or biochemical changes within the cell such that the cell reacts to the presence of the antagonist in the same manner as if the cellular factor was present. Antagonists and inhibitors may include proteins, nucleic acids, carbohydrates, or any other molecules that bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

The term “β-adrenergic receptor antagonist” refers to a chemical compound or entity that is capable of blocking, either partially or completely, the beta (β) type of adrenoreceptors (i.e., receptors of the adrenergic system that respond to catecholamines, especially norepinephrine). Some β-adrenergic receptor antagonists exhibit a degree of specificity for one receptor sybtype (generally β₁); such antagonists are termed “β₁-specific adrenergic receptor antagonists” and “β₂-specific adrenergic receptor antagonists.” The term “β-adrenergic receptor antagonist” refers to chemical compounds that are selective and non-selective antagonists. Examples of β-adrenergic receptor antagonists include, but are not limited to, acebutolol, atenolol, butoxamine, carteolol, esmolol, labetolol, metoprolol, nadolol, penbutolol, propanolol, and timolol. The use of derivatives of known β-adrenergic receptor antagonists is encompassed by the methods of the present invention. Indeed any compound, which functionally behaves as a β-adrenergic receptor antagonist is encompassed by the methods of the present invention.

The terms “angiotensin-converting enzyme inhibitor” or “ACE inhibitor” refer to a chemical compound or entity that is capable of inhibiting, either partially or completely, the enzyme involved in the conversion of the relatively inactive angiotensin I to the active angiotensin II in the rennin-angiotensin system. In addition, the ACE inhibitors concomitantly inhibit the degradation of bradykinin, which likely significantly enhances the antihypertensive effect of the ACE inhibitors. Examples of ACE inhibitors include, but are not limited to, benazepril, captopril, enalopril, fosinopril, lisinopril, quiapril and ramipril. The use of derivatives of known ACE inhibitors is encompassed by the methods of the present invention. Indeed any compound, which functionally behaves as an ACE inhibitor, is encompassed by the methods of the present invention.

As used herein, the term “genotypes” refers to the actual genetic make-up of an organism, while “phenotype” refers to physical traits displayed by an individual. In addition, the “phenotype” is the result of selective expression of the genome (i.e., it is an expression of the cell history and its response to the extracellular environment). Indeed, the human genome contains an estimated 30,000-35,000 genes. In each cell type, only a small (i.e., 10-15%) fraction of these genes are expressed.

### VII. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1

### Materials and Methods

Neonatal rat ventricular myocyte (NRVM) preparation and culture. Hearts were dissected from 1 to 3 day-old Sprague-Dawley rats, minced, and digested with collagenase (Worthington; 600 mg/ml) and pancreatin (Sigma; 1× activity equivalent) in 1× Ads buffer (NaCl [116 mM], HEPES [20 mM; pH 7.4], NaH2PO4 [4.8 mM], KCl [5 mM], MgSO4 [400 mM], and glucose [5.5 mM]). Cells were centrifuged through a step gradient of Percoll (Pharmacia) to separate myocytes from fibroblasts, and the myocyte pool was further enriched by pre-plating for 2 hours to remove adherent fibroblasts from the cell population. Cells were cultured overnight on dishes coated with gelatin (Sigma; 0.2%) in DMEM containing fetal bovine serum (FBS) (10%), L-glutamine (2 mM), and penicillin-streptomycin. After overnight culture, cells were washed with serum-free medium and maintained in DMEM supplemented with Neutridoma-SP (Roche Applied Science), which contains albumin, insulin, transferrin, and other defined organic and inorganic compounds, at either 0.1% or 0.3% v/v, as indicated.

Indirect immunofluorescence. NRVM (2.5×105) were plated on gelatin-coated 6-well dishes. Following the indicated treatments, cells were fixed with formalin (10%) in PBS, permeablized and blocked with PBS containing NP-40 (0.1%) and bovine serum albumin (BSA) (3%), and incubated in the same solution, but with primary antibodies for sarcomeric α-actinin (Sigma, mouse monoclonal; 1:200 dilution) or atrial natriuretic factor (ANF) (Peninsula Labs, rabbit polyclonal; 1:200 dilution). Cells were washed five times with PBS and incubated with fluorescein- or cy3-conjugated secondary antibodies (Jackson Labs, goat polyclonal; 1:200 dilution), followed by a brief incubation with Hoechst dye (Molecular Probes). Cells were washed five times in PBS, one time with water and sequentially covered with mounting solution (Molecular Probes, SlowFade) and glass coverslips. Proteins were visualized with an inverted fluorescence microscope (Olympus, BH-2) at 40× magnification and images captured using a digital camera (Roper Scientific, Photometrics).

ANF competition ELISA and total protein measurements. NRVM were plated on gelatin-coated 96-well dishes (1×104/well). After overnight incubation in the presence of FBS (10%), cells were cultured in serum-free DMEM (200 ml) containing Neutridoma-SP (0.1%) in the absence or presence of PE (Sigma; 20 mM) or FBS (10%) and the indicated concentrations of LMB (LC Laboratories). After 72-hours of stimulation, media supernatants (50 ml) were removed and added to 96-well Immulon II plates (Dynex) that had been previously coated with goat anti mouse Fc fragments (Jackson Labs; 1 mg/ml). Some wells received ANF peptide standards (Phoenix Peptide) in place of culture supernatant. Monoclonal anti-ANF antibody (Biodesign; 50 ng/ml in TBS-T) and biotinylated ANF peptide (Phoenix Peptide; 1 ng/ml in TBS-T) were added to wells (25 ml each) and incubated for 2 hrs at room temperature. Some wells were treated with TBS-T lacking media supernatant or peptide to provide background controls. Wells were washed 5× with TBS-T and exposed to HRP-conjugated streptavidin (Jackson Labs; 100 ml/well of 1:10,000 dilution in TBS-T) for 1 hour at room temperature. Wells were washed 5× with TBS-T and exposed to TMB substrate (Kirkegaard & Perry; 100 ml), which provides a colorimetric readout, for 30 minutes at room temperature. Reactions were terminated with H2SO4 (2N, 100 ml/well) and absorbance at 450 nM detected using a Fusion Plate Reader (Perkin Elmer/Packard). For total protein measurements, cells were washed 5× with PBS and Bradford reagent (Bio-Rad) was added to each well of the plate (100 ml/well). Color development was allowed to proceed for 30 minutes prior to being measured at 595 nm using a micro-titer plate spectrophotometer (Molecular Devices).

α- and β-myosin heavy chain cytoblot assay. NRVM were plated on gelatin-coated 96-well dishes (1×10⁴ cells/well) in DMEM containing L-glutamine (2 mM), penicillin-streptomycin, and charcoal-stripped FBS (10%) lacking thyroid hormone, which is known to induce of α-MyHC expression and coordinately downregulate β-MyHC expression. Following overnight culture, growth medium was replaced with serum-free DMEM supplemented with Neutridoma-SP (0.3% v/v) in the absence or presence of LMB (18.5 nM) and the indicated hypertrophic agonist. Cells were washed two times with PBS and fixed with methanol (30 minutes on ice). Cells were rinsed two times with PBS and blocked with PBS containing BSA (0.1%; 60 minutes at room temperature). Cells were incubated with anti-rat cardiac α-MyHC antibody (ATCC, mouse monoclonal hybridoma supernatant BA-G5) or anti-rat β-MyHC antibody (University of Iowa Developmental Studies Hybridoma Bank, mouse monoclonal hybridoma supernatant) (200 ml/well; 1 hr at room temperature). Cells were washed three times with PBS containing BSA (1%) and incubated with the same solution, but containing HRP-conjugated anti-mouse secondary antibody (Southern Biotech; 1:500 dilution) (1 hr at room temperature). Cells were washed three times with PBS containing BSA (1%), luminol (Pierce) was added, and luminescence detected using a Fusion Plate Reader (Perkin Elmer/Packard).

Cell volume measurements. Three-dimensional measurements of isolates NRVM were conducted using a Coulter Model Z2 instrument (Beckman), as previously described (Said et al., 2004). Briefly, cells in 6-well dishes (2.5×105/well) were washed 1× with PBS and trypsinized (200 ml trypsin; 2 minutes at room temperature). DMEM containing 10% FBS (2 ml) was added to trysinized cells and cells were dispersed by vigorous pipetting. Cells were washed 1× with PBS and transferred to cuvettes containing Isoflow buffer (Coulter; 10 ml). Volume measurements were obtained for 1×104 cells with Coulter aperture diameter limits between 6 and 26 mm. Cell volumes are expressed as mm3.

Toxicity measurements. Adenylate kinase (AK) was measured in culture supernatants employing the bioluminescent ToxiLight kit (BioWhittaker) according to the manufacturer's instructions. Assays were run in a 96-well format and values measured using a Fusion Plate Reader (Perkin Elmer/Packard). Cell viability was also assessed using the LIVE/DEAD Viability/Cytotoxicity Kit (Molecular Probes). This kit employs calcein AM fluorescent dye, which is retained by viable cells, and an ethidium homodimer, which only enters cells with damaged membranes.

RNA analysis. NRVM were plated on gelatin-coated 10 cm dishes (2×10⁶ cells/dish). Following the indicated treatments, RNA was isolated from cardiomyocytes using Trizol Reagent (Gibco/BRL). Total RNA (2 mg) was vacuum blotted onto nitrocellulose membranes (Bio-Rad) using a 96-well format dot blotter (Bio-Rad). Membranes were blocked in 4×SSC containing SDS (1%), 5× Denhardt's Reagent, sodium pyrophosphate (0.05%), and 100 mg/ml sonicated salmon sperm DNA (4 hrs at 500° C.) and incubated with ³²P-end-labeled oligonucleotide probes (1×10⁶ cpm/ml; 14 hrs at 500° C.). Sequences of oligonucleotides were as follows: ANF, 5′-aatgtgaccaagctgcgtgacacaccacaagggcttaggatcttttgcgatctgctcaag-3; a-SK-actin 5′-tggagcaaaacagaatggctggctttaatgcttcaagttttccatttcctttccacaggg-3′; a-MHC 5′-cgaacgtttatgtttattgtggattggccacagcgagggtctgctggagagg-3′; b-MHC 5′-gctttattctgcttccacctaaagggctgttgcaaaggctccaggtctgagggcttc-3′; GAPDH 5′-ggaacatgtagaccatgtagttgaggtcaatgaag-3′). Blots were washed twice with 0.5×SSC containing SDS (0.1%; 10 minutes at 500° C.) and analyzed by autoradiography.

SERCA and MCIP immunoblot analysis. Whole cell proteins extracts were prepared from NRVM using Tris buffer (50 mM, pH 7.5) containing EDTA (5 mM), Triton-X-100 (1%), protease inhibitor cocktail (Complete; Roche), and PMSF (1 mM). Lysates were sonicated briefly and clarified by centrifugation. Protein concentrations were determined by Bradford Assay (Bio-Rad) and 20 mg total protein resolved by SDS-PAGE with gradient gels (Invitrogen; 4%-20% polyacrylamide). Proteins were transferred to nitrocellulose membranes (Bio-Rad) and immunoblotted with anti-SERCA2 antibody (Affinity Bioreagents, mouse monoclonal; 1:1000 dilution) or a peptide antibody directed against the carboxy-terminus of murine MCIP1 (Bush et al., 2004) (rabbit polyclonal; 1:5000 dilution). After washing with TBS-T, membranes were incubated with HRP-conjugated anti-mouse or anti-rabbit secondary antibodies (Southern Biotechnology, 4050; 1:10,000 dilution) and proteins visualized using an enhanced chemiluminescence system (Pierce).

B. Results

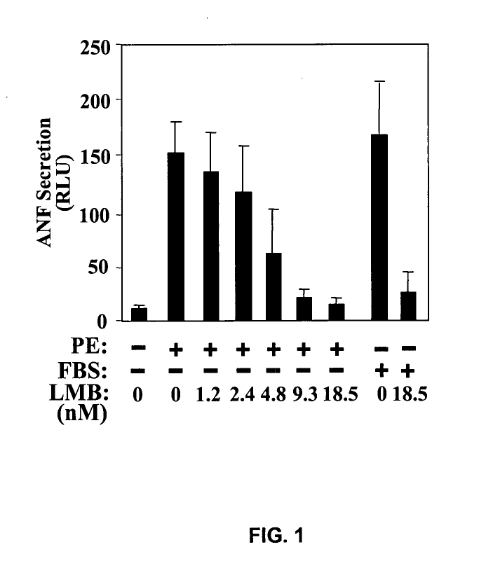

Inhibition of fetal cardiac gene induction by a CRM-1 antagonist. Stress signaling in cardiomyocytes results in up-regulation of the fetal gene program, which is normally silenced in the adult heart. The gene encoding secreted atrial natriuretic factor (ANF) is a prototypical component of this program that is potently upregulated in ventricular myocytes exposed to cues for pathologic cardiac hypertrophy. To begin to investigate the role of CRM-1-dependent nuclear export in the control of cardiac hypertrophy, the inventors assessed ANF protein levels in culture supernatants from primary neonatal rat ventricular myocytes (NRVM) treated with hypertrophic agonists and a CRM-1 antagonist, leptomycin B (LMB). As shown in FIG. 1, ANF expression was dramatically induced in NRVM treated with the alpha-adrenergic agonist phenylephrine (PE) or fetal bovine serum (FBS), both of which trigger hypertrophy. Remarkably, low nanomolar (nM) concentrations of LMB efficiently blocked ANF induction by these hypertrophic agonists.

Agonist-dependent elevation of ANF expression in NRVM can also be examined by indirect immunofluorescence with ANF-specific antibodies. Following stimulation with PE or FBS, prominent ANF protein expression was observed within the secretory pathway, as evidenced by its perinuclear localization (Table 3). Consistent with the reduction in ANF secretion by LMB (FIG. 1), ANF immunostaining was markedly reduced in the presence of this CRM-1 antagonist. LMB also efficiently blocked ANF expression induced by prostaglandin F2a and endothelin-1, which trigger cardiac hypertrophy through distinct receptor signaling events (Table 3).

TABLE 3
Effect of a CRM-1 inhibitor on perinuclear ANF expression in
cardiomyocytes. NRVM were treated with PE (20 mM) or FBS (5%)
in the absence or presence of LMB (18.5 nM), and ANF protein
was detected by indirect immunofluorescence with anti-ANF antibodies.
Agonist induced perinuclear expression of ANF was inhibited
by LMB. +, presence; −, absence.

| Stimulus | LMB (18.5 nM) | Perinuclear ANF |
|---|---|---|
| PE (20 μM) | − | + |
| PE (20 μM) | + | − |
| FBS (5%) | − | + |
| FBS (5%) | + | − |
| PGF2a (10 μM) | − | + |
| PGF2a (10 μM) | + | − |
| ET-1 (50 nM) | − | + |
| ET-1 (50 nM) | + | − |

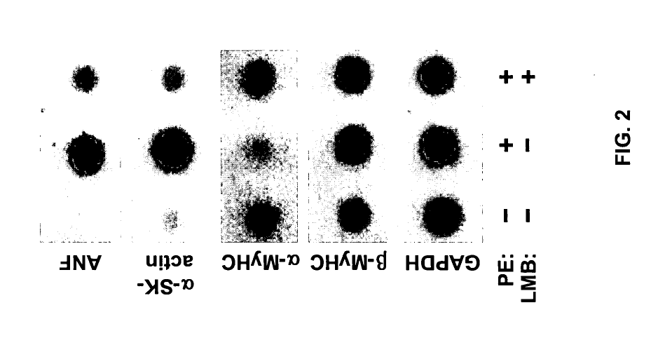

To rule out possible effects of LMB treatment on ANF protein stability, the inventors examined mRNA levels for ANF and other fetal gene markers in cells treated with this CRM-1 antagonist. As shown in FIG. 2, LMB effectively blocked PE-mediated induction of ANF mRNA transcripts, as well as those for another classical fetal cardiac gene marker, α-skeletal actin (α-SK-actin).

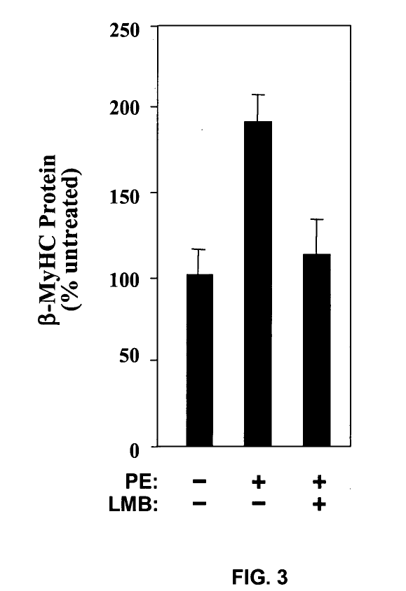

Stress signaling in the heart enhances expression of embryonic β-myosin heavy chain (β-MyHC) and reduces expression of adult α-myosin heavy chain (α-MyHC). Since β-MyHC has slower ATPase activity than α-MyHC, the net outcome of this isoform switch is impaired contractility. As shown in FIGS. 2 and 3, LMB repressed PE-induced β-MyHC mRNA and protein expression and inhibited the subtle downregulation of α-MyHC transcripts. Together, the results demonstrate a role for CRM-1-dependent nuclear export in activation of fetal cardiac genes in response to cues for pathologic hypertrophy.

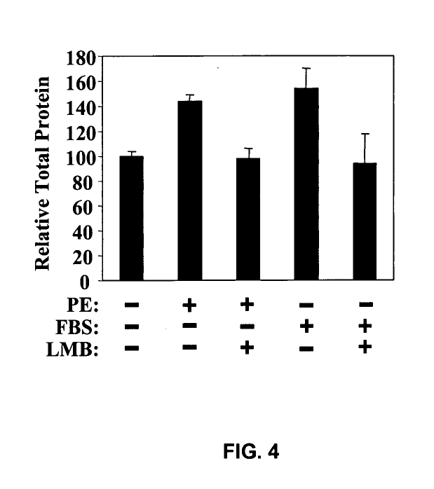

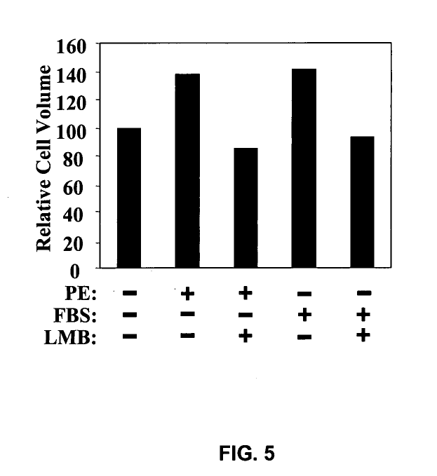

LMB blocks morphologic features of cardiac hypertrophy. In addition to fetal gene induction, cardiac hypertrophy is associated with increased cell size due to enhanced protein synthesis and elevated assembly and organization of sarcomeres. LMB was employed to assess the role of CRM-1 in the regulation of these features of cardiac hypertrophy. Treatment of NRVM for 72 hrs with PE or FBS resulted in an increase in total cellular protein content, which was effectively blocked by LMB (FIG. 4). As shown in FIG. 5, the reduction in protein synthesis observed in LMB-treated cells correlated with diminished cell size, as determined using a Coulter Counter.

NRVM sarcomeres were visualized by indirect immunofluorescence using an antibody directed to sarcomeric α-actinin. Treatment of cells with PE or FBS led to parallel assembly of highly ordered sarcomeres, while sarcomeres from unstimulated cells were disorganized (Table 4). CRM-1 inhibition rendered NRVM unresponsive to the sarcomere-altering effects of the hypertrophic agonists.

TABLE 4
Effect of a CRM-1 inhibitor on NRVM sarcomere organization.
NRVM were treated for 48 hrs with PE (20 mM) or FBS (5%) in
the absence or presence of LMB (18.5 nM). Cells were fixed
and stained for a-actinin to reveal sarcomeres by indirect
immunofluorescence. LMB prevents the enhanced organization
of the sarcomeres in response to hypertrophic agonists. +,
organized sarcomeres; −, disorganized sarcomeres.

| Stimulus | LMB (18.5 nM) | Organized Sarcomeres |
|---|---|---|
| PE (20 μM) | − | + |
| PE (20 μM) | + | − |
| FBS (5%) | − | + |
| FBS (5%) | + | − |

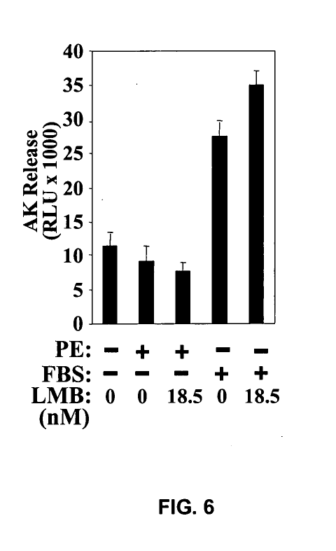

It remained possible that cytotoxicity induced by LMB was being mistaken for anti-hypertrophic activity. To rule out this possibility, the inventors examined whether LMB was toxic to cardiomyocytes by assaying for release of adenylate kinase (AK) into the culture medium as a measurement of cell membrane integrity. As shown in FIG. 6, LMB did not increase AK in the culture medium of cells treated with PE or FBS. Of note, the higher values from FBS-treated cells were a consequence of AK present in serum. As an independent measure of toxicity, cells were stained with calcein AM, which is retained by and fluoresces in viable cells, and ethidium bromide, which only enters cells with compromised membranes. LMB-treated cells retained calcein dye as effectively as control cells, and were not significantly stained by ethidium bromide (Table 5). These results further support the notion that LMB was blocking cardiac hypertrophy by impinging on cellular components required for the process rather than by non-specifically inducing cellular demise.

TABLE 5
Further evidence that CRM-1 inhibition does not diminish cardiomyocyte
viability. NRVM were left untreated or stimulated with PE (20
mM) for 48 hrs in the absence or presence of the indicated concentrations
of LMB. Cells were stained with calcein AM dye, which is retained
by and fluoresces in viable cells, and ethidium bromide (red),
which only enters cells with compromised membranes. +, fluorescence;
−, no fluorescence.

| | | | Ethidium |
|---|---|---|---|
| Stimulus | LMB (nM) | Calcein Fluorescence | Fluorescence |
| PE (20 μM) | 0 | + | − |
| PE (20 μM) | 2.3 | + | − |
| PE (20 μM) | 4.6 | + | − |
| PE (20 μM) | 9.25 | + | − |
| PE (20 μM) | 18.5 | + | − |

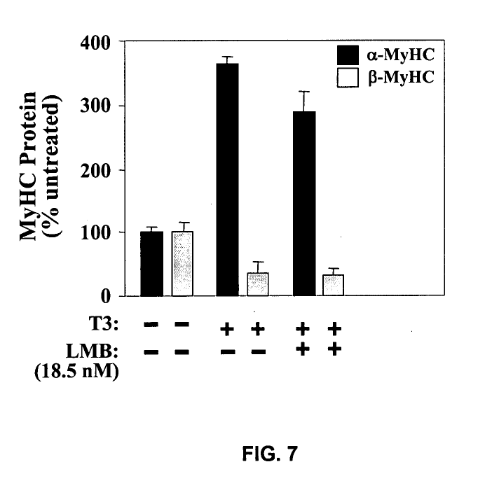

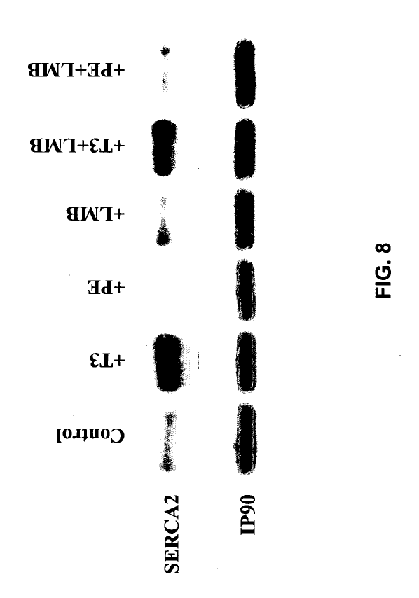

CRM-1 antagonism suppresses pathologic but not physiologic hypertrophy. Growth of the heart that occurs during post-natal development and in response to exercise is referred to as physiologic hypertrophy. The gene program that is activated during physiologic hypertrophy is unique from that observed during pathologic cardiac growth. For example, while the genes for α-MyHC and sarco(endo)plasmic reticulum Ca(2+)-ATPase (SERCA) are repressed during pathologic hypertrophy, they are activated in response to cues for physiologic cardiac growth. Physiologic hypertrophy can be induced in vitro and in vivo by the bioactive form of thyroid hormone, triiodothyronine (T3). To assess the potential role of CRM-1 in the control of physiologic hypertrophy, we exposed NRVM to T3 in the absence or presence of LMB. As shown in FIG. 7, T3 stimulated α-MyHC expression and concomitantly repressed expression of β-MyHC. Remarkably, LMB had no significant effect on this coordinate regulation of MyHC expression. Likewise, inhibition of CRM-1 failed to alter T3-induced SERCA2 expression, and appeared to repress the modest downregulation of this gene that occurs in response to PE (FIG. 8). Taken together, these results demonstrate that LMB potently blocks pathologic cardiac hypertrophy at the level of fetal genes, protein synthesis, cell size and sarcomere assembly, and suggest that CRM-1-dependent nuclear export is dispensable for physiologic hypertrophy.

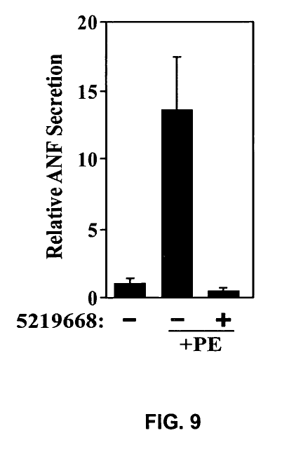

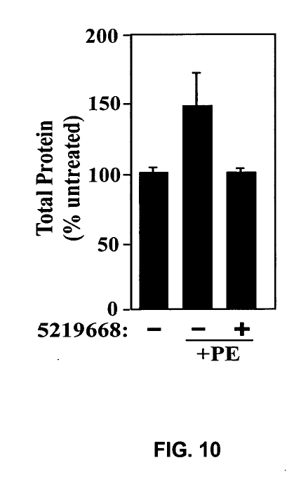

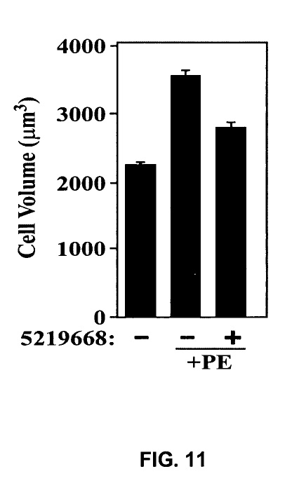

A novel CRM-1 inhibitor blocks agonist-dependent cardiac hypertrophy. To rule out the possibility that inhibition of cardiac hypertrophy by LMB was mediated independently of CRM-1, a novel CRM-1 inhibitor with distinct chemical properties, 2-chloro-N-(6-methyl-2,3,4,9-tetrahydro-1H-carbazol-1-yl)acetamide, was tested for its ability to block agonist-dependent NRVM hypertorphy. Like LMB, this compound, referred to here as 5219668, covalently modifies CRM-1 and acts as a general inhibitor of nuclear export (Kau et al., 2003). As shown in FIG. 9, 5219668 efficiently blocked ANF induction in response to PE. The compound also effectively blocked the increases in protein synthesis and cell size that are normally associated with PE treatment (FIGS. 10 and 11). These findings further support the concept that CRM-1 inhibitors will be beneficial in the context of pathological cardiac hypertrophy.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference:
Ahmed, A., J. Am. Geriatr. Soc., 51(1):123-126 (2003).
Aus. Pat. No. 6,794,700.
Aus. Pat. No. 9,013,101.
Aus. Pat. No. 9,013,201.
Aus. Pat. No. 9,013,401.
Baichwal and Sugden, Gene Transfer, Kucherlapati R, ed., New York, Plenum Press, pp. 117-148, 1986.
Barnes et al., J. Biol. Chem., 272(17):11510-7 (1997).
Bennet et al., Endocrinology, 142:1968-1974 (2001).
Benvenisty and Neshif, Proc. Nat'l Acad. Sci. USA, 83:9551-9555 (1986).
Bhavsar et al., Genomics, 35(1):11-23 (1996).
Bosher et al., Nat. Cell. Biol., 2(2):E31-E36 (2000).
Botinelli et al., Circ. Res. 82:106-115 (1997).
Brand et al., J. Biochem. Cell. Biol., 29:1467-1470 (1997).
Bristow, Cardiology, 92:3-6 (1999).
Bush et al., Proc. Nat'l. Acad. Sci., 101(9):2870-2875 (2004).
Butler et al., Clin. Cancer Res., 7:962-970, (2001).
Butler et al., Cancer Res., 60:5165-5170, (2000).
Caplen et al., Gene, 252:95-105 (2000).
Chang et al., Hepatology, 14:124A (1991).
Chen and Okayama, Mol. Cell Biol., 7:2745-2752 (1987).
Chen et al., Science, 293:1653-1657 (2001).
Clineschmidt et al., J. Pharmacol. Exp. Ther., 235:696-708 (1985).
Coffey et al., Cancer Res., 61:3591-3594, (2001).
Coffin, Retroviridae and Their Replication. In: Virology, Fields et al., eds., Raven Press, New York, pp. 1437-1500 (1990).
Cohen in Ph.D. Thesis (Harvard Univ., Cambridge, Mass.) (1998).
Cohen et al., Proc. Natl. Acad. Sci., 95:14272-14277 (1998).
Cohen and Wittenauer, J. Cardiovasc. Pharmacol., 10: 176-181 (1987).
Colas et al., Nature (London), 380:548-550 (1996).
Conte et al., J. Heart Lung Transplant., 17:679-685 (1998).
Cook et al., Cell, 27:487-496 (1981).
Couch et al., Am. Rev. Resp. Dis., 88:394-403 (1963).
Coupar et al., Gene, 68:1-10 (1988).
Cyert, J. Biol. Chem., 276(24):20805-20808 (2001).
Crabtree, G. R. and Olson, E., Cell, 109:S67-S79 (2002).
Daelmans et al., Proc. Nat'l. Acad. Sci., 99:14440-14445 (2002).
Day, M., et al., J. Neurophysiol., 87:2490-2504 (2002).
De Feo et al., Ital. Heart J., 4:511-513 (2003).
DiBianco, R Am. J. Med., 115:480-488 (2003).
Dolmetsch et al., Nature, 386:855-858 (1997).
Dubensky et al., Proc. Nat'l Acad. Sci. USA, 81:7529-7533 (1984).
Dumcius et al., Medicina, 39:815-822 (2003).
Durand et al., Ann. Med., 27:311-317 (1995).
Eichhorn and Bristow, Circulation, 94:2285-2296 (1996).
Elbashir et al., EMBO, 20(23):6877-6888 (2001).
Emmel et al., Science, 246:1617-1620 (1989).
Epstein, In The Metabolic and Molecular Bases of Inherited Disease: in Down Syndrome (Trisomy 21), Scriver, Beaudet, Valle, (eds), 7th Ed., Vol. 1, pp. 749-794, McGraw-Hill, Inc., New York, 1995.
Eur. Pat. App. No. 0273085
Eur. Pat. No. 1,123,111.
Eur. Pat. No, 1,170,008.
Eur. Pat. No. 1,173,562.
Eur. Pat. No. 1,174,438.
Eur. Pat. No. 1,208,086.
Eur. Pat. No. 1,233,958.
Fabbrizio et al., Oncogene, (1999)
Fechheimer, et al., Proc Nat'l. Acad. Sci. USA 84:8463-8467 (1987).
Ferkol et al., FASEB J., 7:1081-1091 (1993).
Fire et al., Nature, 391:806-811 (1998).
Foguet et al., EMBO, 11:3481-3487 (1992).
Fornerod et al., Cell, 90(6):1051-1060 (1997).
Forster and Symons, Cell, 49:211-220 (1987).
Fraley, et al., Proc Nat'l. Acad. Sci. USA 76:3348-3352 (1979).
Franz et al., Cardoscience, 5(4):235-43 (1994).
Friedmann, Science, 244:1275-1281 (1989).
Fuentes, et al., Genomics, 44:358-361 (1997).
Fuentes, et al., Hum. Mol. Genet., 4:1935-1944 (1995).
Furumai et al., Cancer Res., 62:4916-21 (2002).
Gao et al., J. Biol. Chem., 277:25748-55 (2002).
Gerlach et al., Nature (London), 328:802-805 (1987).
Geyer et al., Proc. Natl. Acad. Sci., 96:8567-8572 (1999).
Ghosh and Bachhawat, In: Liver Diseases, Targeted Diagnosis and Therapy Using Specific Receptors and Ligands. Wu et al., eds., Marcel Dekker, New York, pp. 87-104 (1991).
Ghosh-Choudhury et al., EMBO J., 6:1733-1739 (1987).
Gomez-Foix et al, J. Biol. Chem., 267:25129-25134 (1992).
Goodman and Gilman's “The Pharmacological Basis of Therapeutics.”
Gopal, Mol. Cell Biol., 5:1188-1190 (1985).
Gottlicher et al., EMBO J., 20:6969-78 (2001).
Graham et al., J. Gen. Virol., 36:59-72 (1977).
Graham and Prevec, In: Methods in Molecular Biology: Gene Transfer and Expression Protocol, E. J. Murray, ed., Humana Press, Clifton, N.J., 7:109-128 (1991).
Graham and van der Eb, Virology, 52:456-467 (1973).
Grishok et al., Science, 287:2494-2497 (2000).
Grozinger et al., Proc. Natl. Acad. Sci. USA, 96:4868-4873 (1999).
Grunhaus and Horwitz, Seminar in Virology, 3:237-252 (1992).
Gruver et al., Endocrinology, 133:376-388 (1993).
Han et al., Cancer Research, 60:6068-6074 (2000).
Harland and Weintraub, J. Cell Biol., 101:1094-1099 (1985).
Hermonat and Muzycska, Proc. Nat'l Acad. Sci. USA, 81:6466-6470 (1984).
Hersdorffer et al., DNA Cell Biol., 9:713-723 (1990).
Herz and Gerard, Proc. Nat'l. Acad. Sci. USA 90:2812-2816 (1993).
Hill et al., J. Biol. Chem., 277:10251-10255 (2002).
Hinnebusch et al., J. Nutr., 132:1012-7 (2002).
Ho et al., J. Biol. Chem., 270:19898-19907 (1995).
Hoffmann et al., Bioconjugate Chem., 12:51-55 (2001).
Hoey et al., Immunity, 2:461-472 (1995).
Holaska et al., J. Cell. Biol., 152:127-140 (2001).
Hoppe-Seyler et al., J. Steroid Biochem. Mol. Biol., 78:105-111 (2004).
Horwich, et al., J. Virol., 64:642-650 (1990).
Jans et al., Bioessays, 22(6):532-544 (2000).
Japanese Patent Application No. 2001/348340.
Jones and Shenk, Cell, 13:181-188 (1978).
Joyce, Nature, 338:217-244 (1989).
Jung, Curr. Med. Chem., 8:1505-11 (2001).
Jung et al., J. Med. Chem., 42:4669-4679 (1999).
Jung et al., Med. Chem. Lett., 7:1655-1658(1997).
Kaneda et al., Science, 243:375-378 (1989).
Kao et al., Genes Dev., 14:55-66 (2000).
Kau and Silver, DDT, 8:78-85 (2003).
Karlsson et al., EMBO J., 5:2377-2385 (1986).
Kato et al., J Biol. Chem., 266(6):3361-3364 (1991).
Kau et al., Cancer Cell., 6:463-76 (2003).
Kelly et al., J. Cell Biol., 129(2):383-96 (1995).
Ketting et al., Cell, 99(2):133-141 (1999).
Khan et al., J. Neuroscience, 21:382-391 (2001).
Kim et al., Oncogene, 18:2461-2470 (1999).
Kim and Cook, Proc. Nat'l Acad. Sci. USA, 84:8788-8792 (1987).
Kimura et al., Dev. Growth Differ., 39(3):257-65 (1997).
Kitazomo et al., J. Clinical Endoc. Metabol., 86(7):3430-3435 (2001).
Klein et al., J. Virol., 61:1552-1558 (1987).
Komastsu et al., Cancer Res., 61:4459-4466 (2001).
Kramer et al., Trends in Endoc. Metabolism, 12)7):294-300 (2001).
Kudo et al., Proc. Nat'l. Acad. Sci., 96:9112-9117 (1999).
Kudoh et al., Circ. Res., 80:139-146 (1997).
Kursar et al., Mol. Pharmacol., 46:227-234 (1994).
Le Gal La Salle et al., Science, 259:988-990 (1993).
LaPoint et al., Hypertension, 27:715-722 (1995).
LaPointe, et al., J. Biol. Chem., 263(19):9075-8 (1988).
Launay et al., Nat. Med., 8:1129-1135 (2002).
Lee et al., Am. J. Physiol. Gastrointest. Liver Physiol., 284:G604-G616 (2003).
Levrero et al., Gene, 101:195-202 (1991).
Lin et al., Genetics, 153:1245-1256 (1999).
Lin et al., J. Clin. Invest., 97:2842-2848 (1996).
Macejak and Sarnow, Nature, 353:90-94 (1991).
Mai et al., J. Med. Chem., 45:1778-1784 (2002).
Mann et al., Cell, 33:153-159 (1983).
Manoria and Manoria, J. Indian. Med. Assoc., 101(5):311-312 (2003).
Markowitz et al., J. Virol., 62:1120-1124 (1988).
Marks et al., Clin. Cancer Res., 7:759-760, (2001).
Marks et al., J. Natl. Cancer Inst., 92(15):1210-1216, (2000).
Massa et al., J. Med. Chem., 44:2069-2072 (2001).
Masuda et al., Mol. Cell. Biol., 15:2697-2706 (1995).
McCaffery et al., Science, 262:750-754 (1993).
McKinsey et al., Trends Biochem. Sci., 27:40-47, (2002).
McKinsey et al., Proc. Natl. Acad. Sci. USA, 97:14400-14405, (2000)a.
McKinsey et al., Nature, 408:106-111, (2000)_{b}.
Merck Index, Thirteenth Edition.
Michel and Westhof, J. Mol. Biol., 216:585-610 (1990).
Miyazaki, et al., J Biol. Chem., 271:14567-14571 (1996).
Montgomery et al., Proc. Nat'l. Acad. Sci., 95:15502-15507 (1998).
Moss et al., J. Gen. Physiol., 108(6):473-84 (1996).
Murakami et al., Bioorg. Med. Chem. Lett., 12:2807-2810 (2002).
Nebigil and Maroteaux, Circulation, 108:902-908 (2003).
Nebigil et al., Circulation, 107:3223-3229 (2003).
Negibil et al., Proc. Natl. Acad. Sci., 97:9508-9513 (2000).
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176 (1987).
Nicolau and Sene, Biochim. Biophys. Acta, 721:185-190 (1982).
Northrop et al., Nature, 369:497-502 (1994).
Olson, E. and Williams, R. S., Cell, 101:689-692 (2000).
Olson, E. and Williams, R. S., Bioassays, 22:510-519 (2000).
Olson et al., Dev. Biol., 172(1):2-14 (1995).
Palmiter and Solaro, Basic. Res. Cardiol., 92:63-74 (1997).
Park et al., J. Biol. Chem., 271:20914-20921 (1996).
Paskind et al., Virology, 67:242-248 (1975).
Pazos et al., Eur. J. Pharmacol., 106:539-546 (1984).
PCT Application No. WO 84/03564.
PCT Application No. WO 98/33791.
PCT Application No. WO 99/32619.
PCT Application No. WO 00/44914.
PCT Application No. WO 01/14581.
PCT Application No. WO 01/18045.
PCT Application No. WO 01/36646.
PCT Application No. WO 01/38322.
PCT Application No. WO 01/42437.
PCT Application No. WO 01/68836.
PCT Application No. WO 01/70675.
PCT Application No. WO 02/26696.
PCT Application No. WO 02/26703.
PCT Application No. WO 02/30879.
PCT Application No. WO 02/46129.
PCT Application No. WO 02/46144.
PCT Application No. WO 02/50285.
PCT Application No. WO 02/51842.
Pelletier and Sonenberg, Nature, 334:320-325 (1988).
Perales, et al., Proc. Nat'l Acad. Sci. USA, 91(9):4086-4090 (1994).
Physicians Desk Reference.
Potter et al., Proc. Nat'l Acad. Sci. USA, 81:7161-7165 (1984).
Pritchett et al., EMBO J., 7:4135-4140 (1988).
Racher et al., Biotechnology Techniques, 9:169-174 (1995).
Ragot et al., Nature, 361:647-650 (1993).
Reinhold-Hurek and Shub, Nature, 357:173-176 (1992).
Renan, Radiother. Oncol., 19:197-218 (1990).
Rich et al., Hum. Gene Ther., 4:461-476 (1993).
Ridgeway, Mammalian Expression Vectors, In: Vectors: A Survey of Molecular Cloning Vectors and Their Uses, Rodriguez et al., eds., Stoneham: Butterworth, pp. 467-492, 1988.
Rippe, et al., Mol. Cell Biol., 10:689-695 (1990).
Rooney et al., EMBO J., 13:625-633 (1994).
Rosenfeld, et al., Cell, 68:143-155 (1992).
Rosenfeld, et al., Science, 252:431-434 (1991).
Rothermel et al., Proc. Natl. Acad. Sci. USA, 98:3328-3333 (2001).
Roux et al., Proc. Nat'l Acad. Sci. USA, 86:9079-9083 (1989).
Sadoshima et al., Cell, 75:977-984 (1993)
Sadoshima and Izumo, Circ. Res., 73:424-438 (1993).
Said et al., Biotechniques, 25(3):522-525 (1998).
Sarver et al., Science, 247:1222-1225 (1990).
Saunders et al., Cancer Res., 59-399-409 (1999).
Scanlon et al., Proc. Nat'l Acad. Sci. USA, 88:10591-10595 (1991).
Schmuck et al., FEBS Lett., 342:85-90 (1994).
Sharp et al., Science, 287:2431-2433 (2000).
Sharp, P. A., Genes. Dev., 13:139-141 (1999).
Stemmer and Klee, Biochemistry, 33:6859-6866 (1994).
Stratford-Perricaudet et al., Hum. Gene Ther., 1:241-256 (1990).
Stratford-Perricaudet and Perricaudet, In: Human Gene Transfer, O. Cohen-Haguenauer et al., eds., John Libbey Eurotext, France, pp. 51-61 (1991).
Su et al., Cancer Res., 60:3137-3142 (2000).
Tabara et al., Cell, 99(2):123-132 (1999).
Takahashi et al., Antibiotics, 49:453 (1996).
Taunton et al., Science, 272:371 (1996).
Temin, In: Gene Transfer, Kucherlapati R, ed., New York, Plenum Press, pp. 149-188, 1986.
Tong et al., Nucleic Acids Res., 30:1114-23 (2002).
Top et al., J. Infect. Dis., 124:155-160 (1971).
Tur-Kaspa, et al., Mol. Cell Biol., 6:716-718 (1986).
United States App. 2003/144340
United States App. 2002/61860.
United States App. 2002/65282.
United States App. 2002/103192.
United States App. 2002/256221
U.S. Pat. No. 6,372,957.
U.S. Pat. No. 5,889,136.
U.S. Pat. No. 5,888,773.
U.S. Pat. No. 5,795,715.
U.S. Pat. No. 5,708,158.
U.S. Pat. No. 5,604,251.
U.S. Pat. No. 5,354,855.
U.S. Pat. No. 4,946,778.
U.S. Pat. No. 4,458,066.
U.S. Pat. No. 4,415,732.
U.S. Pat. No. 4,265,874.
U.S. Pat. No. 4,256,108.
U.S. Pat. No. 4,166,452.
Vandebrouck et al., J. Cell. Biol., 158:1089-1096 (2002).
Van den Wyngaert et al., FEBS Lett., 478:77-83 (2000).
Varmus et al., Cell, 25:23-36 (1981).
Vasquez et al., J. Biol. Chem., 278:21649-21654 (2003).
Vennekens et al., Cell Calcium, 31:253-264 (2002).
Vigushin et al., Anticancer Drugs, 13:1-13 (2002).
Vigushin et al., Clinical Cancer Res., 7:971-976 (2001).
Vigushin et al., Cancer Res., 5S (1999).
Vikstrom and Leinwand, Curr. Opin. Cell Biol., 8:97-105 (1996).
Wagner, et al., Proc. Nat'l Acad. Sci. USA 87(9):3410-3414 (1990).
Watkins et al., Hum. Mol. Genet., 4:1721-1727 (1995).
Wincott et al., Nucleic Acids Res., 23:2677-2684 (1995).
Wolfe et al., Nature, 385:172-176 (1997).
Wong et al., Gene, 10:87-94 (1980).
Workman and Kingston, Annu. Rev. Biochem., 67:545-579 (1998).
Wu and Wu, Adv. Drug Delivery Rev., 12:159-167 (1993).
Wu and Wu, Biochemistry, 27:887-892 (1988).
Wu and Wu, J. Biol. Chem., 262:4429-4432 (1987).
Xu et al., Proc. Natl. Acad. Sci., 94:12473-12478 (1997).
Yamauchi-Takihara, et al., Proc. Nat'l Acad. Sci. USA, 86(10):3504-8 (1989).
Yamazaki et al., Circulation, 95:1260-1268 (1997).
Yamano et al., Amer. Soc. Gene Ther. (2000).
Yang, et al., Proc Nat'l Acad. Sci. USA, 87:9568-9572 (1990).
Young et al., Handbook of Applied Therapeutics, 7.1-7.12 and 9.1-9.10 (1989).
Yue et al., Nature, 410:705-709 (2001).
Zelenin et al., FEBS Lett., 280:94-96 (1991).
Zhang et al., Cell, 110:479-488, (2002).
Zhou et al., Proc. Natl. Acad. Sci., 98:10572-10577 (2001).
Ziober and Kramer, J. Bio. Chem., 271(37):22915-22 (1996).
Zou et al., J. Biol. Chem., 271:33592-33597 (1996).

1. A method of treating pathologic cardiac hypertrophy or heart failure comprising:
(a) identifying a patient having pathologic cardiac hypertrophy or heart failure;
(b) selecting a known non-selective inhibitor of protein nuclear export; and
(c) administering said inhibitor to said patient.

2. The method of claim 1, wherein said non-selective inhibitor binds to an export protein.

3. The method of claim 1, wherein said inhibitor of nuclear export is selected from the group consisting of peptide aptamers, leptomycin B, valtrate, callystatin A, polyketides, PKF050-638, STI571, staurosporine and staurosporine-related compounds

4. The method of claim 1, wherein administering comprises intravenous administration of said inhibitor of nuclear export.

5. The method of claim 1, wherein administering comprises oral, transdermal, sustained release, suppository, or sublingual administration.

6. The method of claim 1, further comprising administering to said patient a second therapeutic regimen.

7. The method of claim 6 wherein said second therapeutic regimen is selected from the group consisting of a beta blocker, an iontrope, a diuretic, a phosphodiesterase inhibitor, an endothelin receptor antagonist, ACE-I, AII antagonist, a Ca⁺⁺-blocker, and HDAC inhibitor, a TRP channel inhibitor, a 5-HT2 receptor agonist, or a 5-HT2 receptor antagonist.

8. The method of claim 6, wherein said second therapeutic regimen is administered at the same time as said inhibitor of nuclear export.

9. The method of claim 6, wherein said second therapeutic regimen is administered either before or after said inhibitor of nuclear export.

10. The method of claim 1, wherein treating comprises improving one or more symptoms of cardiac hypertrophy.

11. The method of claim 10, wherein said one or more symptoms comprises increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

12. The method of claim 1, wherein treating comprises improving one or more symptoms of heart failure.

13. The method of claim 12, wherein said one or more symptoms comprises progressive remodeling, ventricular dilation, decreased cardiac output, impaired pump performance, arrhythmia, fibrosis, necrosis, energy starvation, and apoptosis.

14. The method of claim 1, wherein said inhibitor of nuclear export is a reversible inhibitor of nuclear export.

15. A method of preventing pathologic cardiac hypertrophy or heart failure comprising:
(a) identifying a patient at risk of developing pathologic cardiac hypertrophy or heart failure; and
(b) selecting a known non-selective inhibitor of protein nuclear export; and
(c) administering said inhibitor to said patient.

16. The method of claim 15, wherein said non-selective inhibitor binds to an export protein.

17. The method of claim 15, wherein said inhibitor of nuclear export is selected from the group consisting of of peptide aptamers, leptomycin B, valtrate, callystatin A, polyketides, PKF050-638, STI571, staurosporine and staurosporine-related compounds

18. The method of claim 15, wherein administering comprises intravenous administration of said inhibitor of nuclear export.

19. The method of claim 15, wherein administering comprises oral, transdermal, sustained release, suppository, or sublingual administration.

20. The method of claim 15, wherein the patient at risk may exhibit one or more symptom comprising long standing uncontrolled hypertension, uncorrected valvular disease, chronic angina and/or recent myocardial infarction.

21. The method of claim 15, wherein said inhibitor of nuclear export is a reversible inhibitor of nuclear export.

22. The method of claim 15, further comprising administering to said patient a second therapeutic regimen.

23. The method of claim 22 wherein said second therapeutic regimen is selected from the group consisting of a beta blocker, an iontrope, a diuretic, a phosphodiesterase inhibitor, an endothelin receptor antagonist, ACE-I, AII antagonist, a Ca⁺⁺-blocker, and HDAC inhibitor, a TRP channel inhibitor, a 5-HT2 receptor agonist, or a 5-HT2 receptor antagonist.
